(19) United States
(12) Patent Application Publication
(10) Pub. No.: US 2015/0194034 A1
(43) Pub. Date: Jul. 9, 2015

(54) SYSTEMS AND METHODS FOR DETECTING AND/OR RESPONDING TO INCAPACITATED PERSON USING VIDEO MOTION ANALYTICS

(71) Applicant: NEBULYS TECHNOLOGIES, INC., Frederick, MD (US)

(72) Inventors: Paul C. SHIM, McLean, VA (US); Jiseok LEE, McLean, VA (US); Kyung-Hee KIM, McLean, VA (US)

(21) Appl. No.: 14/587,949

(22) Filed: Dec. 31, 2014

Related U.S. Application Data
(60) Provisional application No. 61/923,447, filed on Jan. 3, 2014.

(51) Int. Cl. G08B 21/04; G06T 7/20; G06K 9/00; H04N 7/18; A61B 5/01; A61B 7/04; A61B 5/117; A61B 5/11; A61B 5/0205; H04N 5/30; A61B 5/00

(52) U.S. Cl. CPC G08B 21/0476; H04N 5/30; G06T 7/20; G06K 9/00342; G08B 21/0415; H04N 7/183; A61B 5/0077; A61B 7/04; A61B 5/7405; A61B 5/1176; A61B 5/7282; A61B 5/747; A61B 5/749; A61B 5/1128; A61B 5/1117; A61B 5/02055; A61B 5/015; A61B 5/0008; A61B 5/0013; G06T 2207/10016; G06T 2207/30196; G06T 2207/30048; G06K 2209/05; A61B 2560/0475; A61B 2576/00; A61B 5/02405

(57) ABSTRACT

The present disclosure provides systems and methods for monitoring an area by an imaging system and detecting an incapacitated person in the monitored area. An incapacitated person monitoring apparatus may include a processing system configured to receive video data from at least one camera, the video data including a plurality of sequentially captured images. The processing system may be further configured to detect a body of a person in the images, and determine whether the detected body of the person in the images satisfies one or more first conditions identifying an incapacitated person. When the body of the person satisfied one or more first conditions, a notification may be transmitted to one or more notification locations.

### CROSS-REFERENCES TO RELATED APPLICATIONS

The present application claims priority to U.S. Provisional Application No. 61/923,447, filed on Jan. 3, 2014, the entirety of which is incorporated by reference herein.

### BACKGROUND

The subject matter of this application is directed to detecting an incapacitated person and more specifically to detecting and responding to the incapacitated person using video and/or audio analytics combined with the automated audio and visual signal confirmation methods.

Individuals with certain medical conditions or individuals in their old age are traditionally put into communities where they can be regularly monitored by medical personnel. In these communities (e.g., nursing homes or assisted living facilities) the medical personnel can come by on a regular basis to make sure that the individual is feeling well. However, such regular visits by the medical personnel can disturb an individual wanting privacy. Others want to keep their independence and would prefer to remain in their own residence instead of moving to such communities.

To allow the individuals to live at their own residence, some have proposed to use devices that are carried by the individual or attached to the individual's body. Some of these devices include a help button that is activated by the individual to request help. Other devices request help when sensors in the wearable device detect that the individual has fallen. However, with certain medical conditions, the individual is not able to activate the help button. In addition, existing devices must be within reach of the individual or must be attached to the individual in order for the devices to be able to detect when help is needed.

### BRIEF DESCRIPTION OF THE DRAWINGS

So that features of the present invention can be understood, a number of drawings are described below. It is to be noted, however, that the appended drawings illustrate only particular embodiments of the invention and are therefore not to be considered limiting of its scope, for the invention may encompass other equally effective embodiments.

FIG. 1 illustrates an incapacitated person monitoring system according to an embodiment of the present disclosure.

FIG. 2 illustrates an incapacitated person monitoring device according to an embodiment of the present disclosure.

FIG. 3 illustrates an arrangement of the incapacitated person monitoring devices inside and outside of a building according to an embodiment of the present disclosure.

FIG. 4 illustrates a method for monitoring a predetermined area for an incapacitated person according to an embodiment of the present disclosure.

FIG. 5 illustrates a method of detecting an incapacitated person according to an embodiment of the present disclosure.

FIG. 6 illustrates a method for responding to a notification of an incapacitated person received from a monitoring system according to an embodiment of the present disclosure.

### DETAILED DESCRIPTION

Embodiments of the present disclosure provide systems and methods for monitoring an area by an imaging system and detecting an incapacitated person in the monitored area. An incapacitated person may include a person who is physically incapacitated (e.g., unable to move or respond).

The monitoring system may include one or more cameras with audio capabilities capturing a sequence of images of areas where a person is expected to spend a majority of his or her time. The captured images along with sounds may be processed to detect an anomaly when a person becomes ill and incapacitated. Examples include detecting sharp movements representing a fall, an irregular heart rate of the person, abnormal loud sounds similar to person body hitting the ground and/or abnormal body temperature. When such anomalies are detected, an automated and prerecorded speaker may be configured to repeatedly inquire if the person is in need of medical attention. A response from the person may be received by analyzing images captured by the camera and/or sound captured by a microphone. If there is no movement and/or no vocal response, a notification may be transmitted to another location (e.g., a designated person and/or an emergency operator) indicating that medical assistance is needed.

The designated person and/or the emergency operator who receive the notification may be given access to the video feed of the camera to determine the severity of the situation by a live person, when the above automated response system determines that the person is in need of medical attention. The monitoring system may also be configured for vocal communication with the incapacitated person via a speaker located in the vicinity of the camera.

When the fall or sharp movement is not identified as being severe, the monitoring system may prompt for a vocal response from the user that confirms or denies an emergency incident. In some embodiments, the person may be prompted for a specific physical response (e.g., waving hands) to identify whether medical assistance is needed. The responses to identify seriousness may be customized by the person. The person's response may also grant or deny access of the video and/or the audio feed to the designated person and/or the emergency operator.

Embodiments of the present disclosure provide systems and methods that do not require the wearing of any device by the person being monitored and do not unnecessarily intrude the person's privacy. The systems and method may be provided within a person's residence and may automatically alert emergency personnel when assistance is needed. Thus, the system is able to automatically request assistance even when the person cannot reach a phone or cannot speak and/or move. Preprogramed body motion signals (e.g., hand motion) or pre-recorded voice commands may also be used to request for assistance.

Other objectives and advantages of the present invention will become apparent to the reader and it is intended that these objectives and advantages are within the scope of the present disclosure.

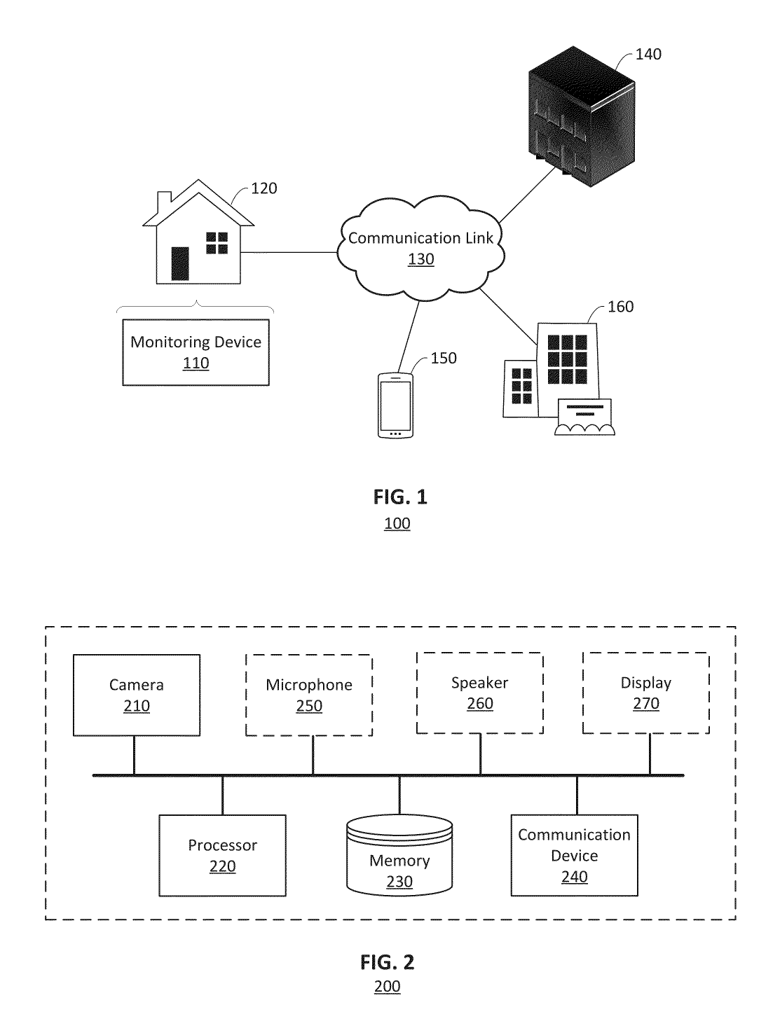

FIG. 1 illustrates an incapacitated person monitoring system 100 according to an embodiment of the present disclosure. The incapacitated person monitoring system 100 may include a stationary monitoring device 110 provided at one or more predetermined locations (e.g., rooms of a residence 120 of a person to be monitored). As discussed in more detail below, the monitoring system 110 may include one or more cameras and/or sensors to monitor the predetermined location and detect an incapacitated person. When an incapacitated person is detected, the monitoring device 110 may transmit a notification indicating detection of the incapacitated person. The notification may be transmitted over a communication link 130 to a monitoring center 140, a designated location 160 (e.g., hospital), and/or a mobile device 150. The device 110 may connect to the communication link 130 via a wired copper line, Bluetooth, Zigbee, Wi-Fi, or other wired and/or wireless connectivity means. The communication link 130 may connect to the monitoring units 140, 150, and 160 via a dial up telephone line, cable/dsl/satellite based internet, and/or 3G/4G/CDMA/LTE capable devices.

When an incapacitated person is detected, the monitoring device 110 may request a response from the incapacitated person to determine whether the person needs assistance. For example, the monitoring device 110 may output a pre-recorded question and detect a response from the incapacitated person. Based on the results of the response or no response within a given time period, the monitoring device 110 may send a notification indicating detection of the incapacitated person to one or more other locations.

In one embodiment, after the notification indicating detection of the incapacitated person is transmitted, the monitoring device 110 may establish a communication channel with the monitoring center 140, the designated location 160, and/or the mobile device 150. The established communication channel may allow for additional information to be received from the monitoring device 110 and/or may allow for direct communication with the incapacitated person. For example, an operator at the monitoring center 140, the designated location 160, or the mobile device 150 may be provided with video signals and/or audio signals captured by the monitoring device 110. The monitoring device 110 may also receive video signals and/or audio signals from the monitoring center 140, the designated location 160, or the designated mobile device 150. Thus, after an incapacitated person is detected by the monitoring device 110, based on the notification and/or the additional information received from the monitoring device, the person at the monitoring center 140, the designated location 160, or the mobile device 150 may request for appropriate emergency personnel to response to the identified emergency. In other embodiments, the monitoring device 110 or other devices at the monitoring center 140 or the designated location 160 may automatically notify emergency personnel of the incapacitated person when certain conditions are satisfied.

FIG. 2 illustrates an incapacitated person monitoring device 200 according to an embodiment of the present disclosure. The incapacitated person monitoring device 200 may include a camera 210, a processor 220, memory 230, and a communication device 240. The incapacitated person monitoring device 200 may also include a microphone 250, a speaker 260, and a display 270. The components of the incapacitated person monitoring device 200 may be communicatively coupled to one or more other components of the incapacitated person monitoring device 200. For example, the camera 210, the microphone 250, the speaker 260, and the display 270 may each be directly coupled to the processor 220 and/or the memory 230.

The camera 210 may be configured to capture video comprising a plurality of images of a predetermined scene. For example, the camera 210 may be disposed in a predetermined location of a room to capture images of a predetermined portion of the room (e.g., the whole room or a partial area of the room). The camera 210 may include a wide angle lens and/or a plurality of imaging sensors arranged to capture a wider area or multiple locations of a designated area. In one embodiment, the camera 210 may be an omnidirectional camera with a 360-degree field of view. In another embodiment, the camera 210 may be a pan-tilt-zoom (PTZ) camera. The PTZ camera may be configured to automatically pan, tilt or zoom at predetermined intervals of time, follow a person located in the room, and/or be controlled in response to control signals (e.g., from the processor or a mobile device).

The processor 220 may include multiple processors configured to receive data captured by the camera 210, signals or data captured by the microphone 250, and data stored in the memory 230. The processor 220 may process the received data, and based on the results, issue instructions to other components. For example, the processor 220 may analyze the video stream received from the camera 210, and based on the results of the analysis, send a notification (e.g., via the communication device) to notify detection of an incapacitated person. The processor 220 may also issue signals to control the camera 210, send pre-recorded instructions or questions to the speaker 260, and/or send text or images to the display 270.

The memory 230 may store one or more programs and information of the incapacitated person monitoring device 200. The programs may provide instructions for analyzing the received data (e.g., images and sound signal), and instructions for performing operations when predetermined conditions are detected. The information in the memory 230 may include location information at which the incapacitated person monitoring device 200 is located, person(s) with which the incapacitated person monitoring device 200 is associated, conditions indicating an incapacitated person, pre-recorded sound commands, customized/trained personal responses, and identification of locations and/or persons to notify when an incapacitated person is detected.

The communication device 240 may be configured to communicate with one or more devices that are disparately located from the incapacitated person monitoring device 200. The communication device 240 may be configured to communicate with one or more locations and/or individuals identified in the memory when predetermined conditions are satisfied (e.g., person is incapacitated and not responsive to questions). The communication device 240 may also allow for video data and sound data to be transmitted between the incapacitated person monitoring device 200 and devices associated with one or more locations and/or people identified in the memory. According to one embodiment, the communication device 240 may also communicate with other cameras in close proximity to the incapacitated person monitoring device 200 (e.g., within the same building).

The display 270 may be coupled to the processor and may be configured to communicate with the incapacitated person. For example, the same commands that are issued via the speaker 260 may be displayed in text form on the display 270. The display 270 may include a touch panel display that is configured to receive user inputs. The display 270 may provide a user interface for inputting user information (e.g., name, age, medical conditions, address, doctor information, relative information, and/or emergency contact information). The user interface may also provide for a user to designate who may have authorization to access data captured by the camera 210 and/or the microphone 250 and under what conditions. For example, the user may designate that a close relative always has authority to access the data being captured by the camera 210 and the microphone 250, while an administrator at a monitoring station may only have access to the data being captured by the camera and the microphone when predetermined conditions are satisfied (e.g., person is detected as being incapacitated and not responsive to pre-recorded questions). In one embodiment, the user may provide each designated person access to a different combination of cameras and/or microphones.

In one embodiment, the components of the incapacitated person monitoring device 200, shown in FIG. 2, may all be provided in a common housing. In another embodiment, one or more components may be provided outside of the common housing. For example, the camera 210, microphone 250, and speaker 260 may be provided in a common housing that is disparately located form the processor 220, memory 230, and the communication device 240. In one embodiment, the speaker 260 may be physically separate from the camera 210 but still be coupled to the monitoring system 200 via wires or wirelessly. In one embodiment, the microphone may be built into the camera.

The incapacitated person monitoring device 200 may be stationary. For example, the incapacitated person monitoring device 200 may be mounted to a wall or ceiling of a residence or other places being monitored. In another embodiment, the incapacitated person monitoring device 200 may be provided on a mobile motorized platform that is configured to follow the person in the vicinity of the residence.

While not shown in FIG. 2, the incapacitated person monitoring device 200 may include additional sensors. The sensors may include motion sensors and/or thermal sensors. The data from these sensors may identify presence of a person and/or when a person is incapacitated. In one embodiment, the camera 210 may be a thermographic camera, such as infrared camera or a thermal imaging camera. The incapacitated person monitoring device 200 may be passive until the thermographic camera detects a person due to the light radiating from the body in the images.

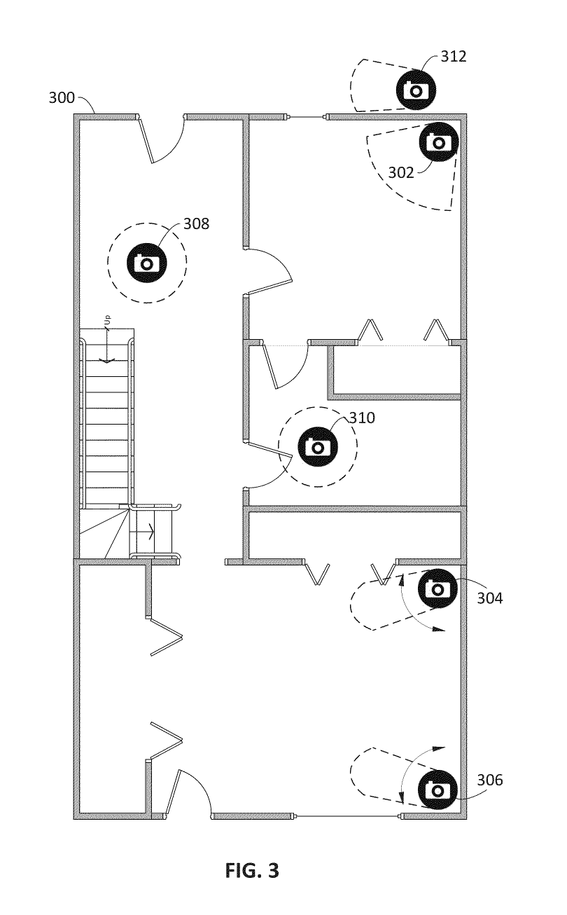

FIG. 3 illustrates an arrangement of the incapacitated person monitoring devices 302-312 inside and outside of a building 300 according to an embodiment of the present disclosure. The building 300 may be a residence, an office building, a healthcare facility, a nursing home, or a senior living facility. As shown in FIG. 3, a plurality monitoring devices 302-312 may be located throughout the building 300. Monitoring devices 302-310 may be located inside the building 300 and monitoring device 312 may be located outside of the building 300. The monitoring devices 302-310 may be mounted on walls or on the ceiling to avoid other objects located within the room from obstructing the field of view of the monitoring devices 302-310.

The monitoring devices 302-312 may be positioned at locations providing the maximum amount of coverage by the cameras associated with the monitoring devices 302-312. For example, the monitoring device 302 may be positioned in a corner of the room to capture a scene of the whole room. The direction of the angle of view of the camera associated with each of the monitoring devices 302-312 is shown with dashes. In one embodiment a plurality of incapacitated person monitoring devices 304 and 306 may be provided within the same room. The cameras in the incapacitated person monitoring devices 304 and 306 may be provided in a PTZ mechanism to provide greater coverage of the room. The incapacitated person monitoring devices 308 and 310 may include omnidirectional cameras providing 360 degrees field of view.

In FIG. 3, each of the incapacitated person monitoring devices 302-312 may be a complete monitoring system able to operate individually. In another embodiment, one or more of the incapacitated person monitoring devices 302-312 may be coupled to each other to provide a network of monitoring devices. In one embodiment, one of the incapacitated person monitoring devices (e.g., the incapacitated person monitoring device 302) may be a master monitoring device and the remaining incapacitated person monitoring devices 304-312 may be slave devices. The slave devices do not have to include all of the components of the master monitoring device. In one embodiment, the slave devices may each include a camera and a communication device configured to communicate with the master monitoring device. The slave device may also include a microphone and/or a speaker. The master monitoring device may receive data from the slave devices and process the data to determine if the person is incapacitated based on the data received from the slave device. The data from the slave devices may be received by the processing system via wires or wirelessly. The master device may also transmit data to the slave devices. The data transmitted to the slave device may include a request for a response from the incapacitated person.

In one embodiment, one or more of the incapacitated person monitoring devices may include a sensor to monitor for motion. When motion is detected by a sensor associated with one of the incapacitated person monitoring devices, the camera of the respective incapacitated person monitoring device may be activated to capture images and to transmit the images to the processor (e.g., processor of the master device). In one embodiment, motion may be detected within a room by capturing images at predetermined intervals of time (e.g., every 5 minutes) and comparing the captured image to a previously captured image to determine if there is motion. When such motion is detected, the camera may receive control signal to start capturing and transmitting a video stream including a plurality of sequentially captured images.

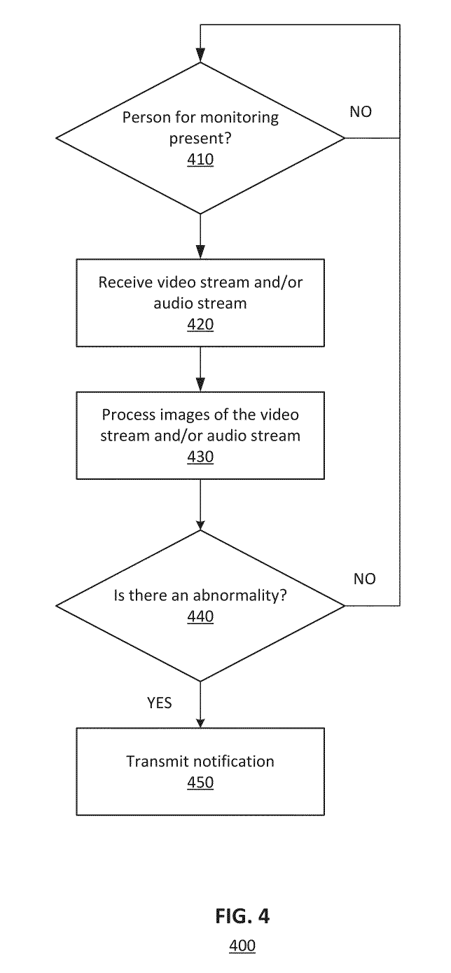

FIG. 4 illustrates a method 400 for monitoring a predetermined area for an incapacitated person according to an embodiment of the present disclosure. The method 400 may include determining if a person for monitoring is present 410, when the person is present, receiving a video stream 420, processing images of the video stream 430 to determine if there are any abnormalities (e.g., the person is incapacitated) 440, and when it is determined that there is an abnormality, transmitting notification of the incapacitated person 450.

Determining if the person for monitoring is present 410 may be performed based on data received from a motion sensor, a microphone, an infrared camera and/or from the camera. The motion sensor may be configured to detect when the person enters a predetermined area and to transmit a signal indicating presence of the person to a processing system. The microphone may be configured to monitor the sound and to transmit a signal to the processor when sound above a predetermined level is detected. Based on the received signal(s), the processing system may activate the camera to capture the video stream. In one embodiment, the motion sensor and/or the microphone may be directly coupled to the camera and may activate the camera to capture the video stream when presence of the person is detected.

In one embodiment, the sound captured by the microphone may be transmitted to the processing system to be analyzed to determine if a person is present within the vicinity of the microphone and the camera. The processing system may analyze the received sound to determine if a person is speaking or if noise above a predetermined threshold is present in the received sound.

In one embodiment, the images captured by the camera may be analyzed to determine whether there is motion in the area being monitored by the camera. Images captured at predetermined intervals may be compared to each other to determine that there is presence of a person in the area being monitored. In one embodiment, the camera or the processing system may be configured to perform face recognition to determine presence of a specific person. In another embodiment, the audio signal captured by the microphone may be analyzed to determine whether there is someone present in the area being monitored by the microphone and/or the monitoring device.

When the presences of a person is detected (YES in step 410), the processing system may receive the video stream from the camera and/or the audio stream from the microphone 420. The video stream may include a plurality of sequentially captured images of the predetermined scene. The video stream may be received by the processing system as long as there is presence of a person detected with the monitoring area and/or for a predetermined period of time after the presence of the person is not detected (e.g., after the person leaves the room).

The received video stream and/or audio stream may be processed 430 to determine if there are any abnormalities suggesting a need of medical attention 440. Determining whether there are any abnormalities 440 may include determining whether there is an incapacitated person in need of medical attention. Processing the received video stream may include following the body movement of the person and distinguishing the body from the background by identifying the anatomical position of the body and its movement in the field of view.

Determining whether abnormities of the person may include analyzing the images to detect predefined body motion (e.g., hand motion), a fall or sudden motion, irregular heart rate, changes in body temperature, or lack of regular chest motion. To detect the predefined body motion, image processing techniques may be performed to compare body motion of the person to predefined body motions stored in the memory (e.g., by the manufacturer of the monitoring device of by the person to be monitored). The predefined body motions may be recorded and stored in memory. The monitoring device may have a calibration mode in which a user is guided by instructions to provide the predefined body motions and/or predefined voice commands.

The fall or sudden motion may be detected by computing motion vector(s) in subsequent images (e.g., between a predefined number of images) and comparing the direction and/or magnitude of the motion vector(s) to predefined values to determine if a fall or sudden motion is present in the captured images. In another embodiment, the incapacitated person may be detected when motion vectors that should be present are not present in subsequent images. For example, an incapacitated person may be detected when the direction of the motion vector is in a downward direction and then the motion vector(s) are no longer present in the captured images. In one embodiment, the incapacitated person may be detected when the motion stops for at least a predetermined period of time.

A thermographic camera may be included in the monitoring system to monitor the heart rate and/or body temperature changes. For example, the processing system may receive images captured by the thermographic camera and determine when the heart rate of the person exceed predetermined acceptable range or when the heart rate is irregular. The processing system may receive images from the thermographic camera and determine whether a temperature drop or a temperature rise exceeds predetermined low threshold or a predetermined high threshold, respectively. In some embodiments, the thermographic camera may be a high frame rate thermographic camera to allow for accurate detection of the person's heart rate.

When it is determined that there is no abnormality (e.g., person is not incapacitated) (NO in step 440), the system may continue to monitor for presence of the person in the scene 410 and/or to receive additional video stream 420. When it is determined that there is an abnormality (e.g., the person is incapacitated) (YES in step 440), a notification requesting for assistance may be transmitted 450 to another location (e.g., a mobile device, a monitoring station, and/or an emergency center). The notification may be retransmitted at predetermined intervals until a response is received or until the monitoring system is reset. The notification, may include identification information stored in the memory. The identification information may include the name, address, medical conditions, emergency contact and other information for the person associated with the monitoring system. The notification may include when the person was incapacitated, location of the incapacitated person in the residence (e.g., location of the monitoring system or the camera used to detect the incapacitated person), and/or one or more images captured by the camera (e.g., one image right before the incapacitated person is detected and one image right after the incapacitated person is detected). In one embodiment, the notification may include the captured video stream.

In one embodiment, the signals captured by the microphone in the sound stream may be analyzed to determine an incapacitated person. The sound signals generated by the microphone may be received by the process to interpret human speak, and look for the particular commands to trigger a particular response for the apparatus. For example, a person can shout a particular voice command that is designated as a distress call by the monitoring system. Thus, the microphone may be used to relay anything the user might need to say in response to any inquiries, false alarms or to be descriptive of a situation. Processing the received audio stream may include determining whether there are signals in the audio stream that exceed preset limits or whether signals are not present in the audio stream when there should be at least some presence of signals. Thus, the monitoring system may request for help even when the person who is incapacitated is not within the field of view of the camera but is with the range of the microphone. The microphone may be configured to continuously capture sound.

In one embodiment, the camera may be powered at all times to enable detection of the presence of the person and the incapacitated person. In other embodiment, the camera may be activated and powered only when the presence of the person for monitoring is detected (e.g., by a motion sensor or an infrared camera). In this embodiment, the motion sensor or the infrared camera may be powered at all times.

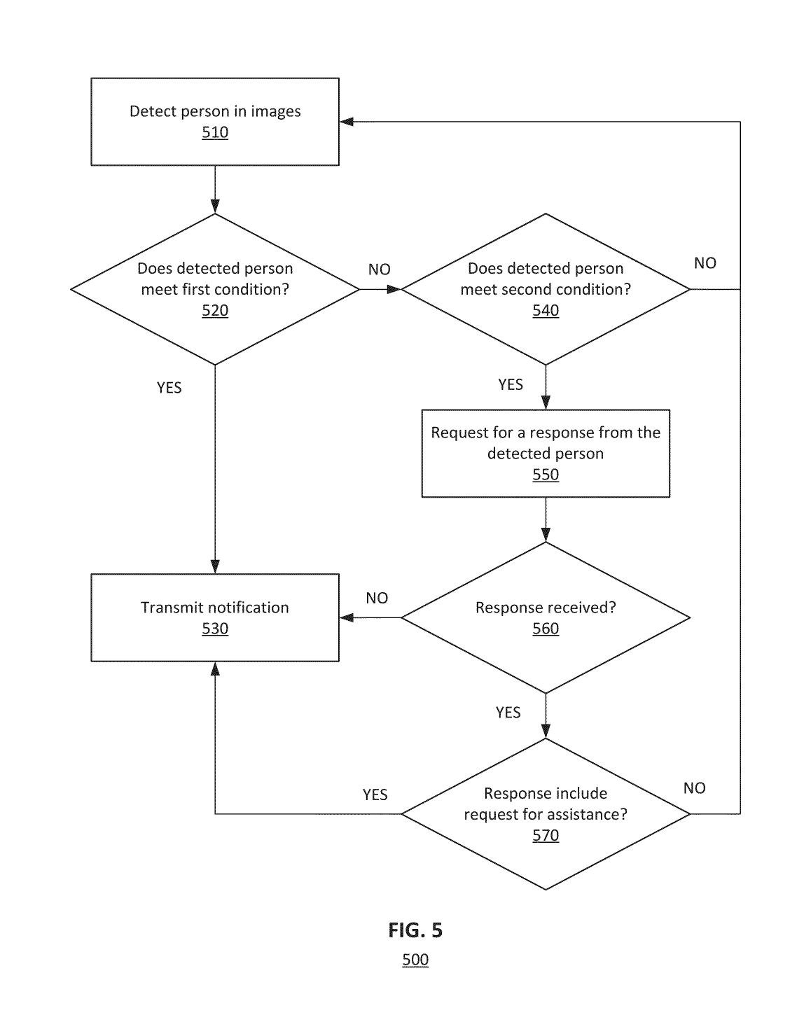

FIG. 5 illustrates a method 500 of detecting an incapacitated person according to an embodiment of the present disclosure. The method 500 may automatically request for assistance when the person is determined in need of immediate medical condition or when the person does not respond within a predetermined period of time. When the incapacitated person is detected, the method 500 may include requesting a response from the incapacitated person to determine whether the assistance is needed. The method 500 may include (1) a first set of conditions which, when detected in the captured images, will automatically trigger transmission of a notification and (2) a second set of conditions which will initiate a request for a response from the detected person to determine if a notification should be transmitted.

As shown in FIG. 5, the method 500 may include detecting a person in the images 510. The camera and the processor may be configured to provide a camera detection system that detects and/or tracks movement of the body. When the person is detected in the images, the images may be processed 520 and 540 to determine if the person is incapacitated (i.e., meets one or more of the first conditions or the second conditions stored in memory). If the detected person meets one or more of the first conditions (YES in step 520), a notification requesting help may be automatically transmitted. The first conditions may include conditions identifying that a person is incapacitated, not able to respond, and needs immediate help. For example, the first conditions may include a person falling and not moving for a predetermined period of time or a stopped heart rate.

If one of the first conditions is not satisfied (NO in step 520), a determination may be made as to whether the detected person meets one or more of the second conditions. The second conditions may include situations where a person is incapacitated but may not be in need of immediate assistance. For example, the second conditions may include a person falling but still able to move or the person having an irregular heart rate.

If one of the second conditions is satisfied (YES in step 540), then a response from the detected person may be requested 550. The request may be a pre-recorded audio request, a musical tune or other sound generated by the speakers. In one embodiment, a request may be made by displaying a message on a display screen.

After the request for a response is transmitted, a determination may be made whether the detected person responded to the request 560. If the detected person does not respond to the request (NO step 560), a notification requesting help may be automatically transmitted 530. If the person responds, the response may be analyzed to determine if the response includes a request for assistance 570. If the response includes a request for assistance, a notification requesting help may be transmitted 530.

The response from the detected person may be detected by monitoring the sound captured by the microphone to detect a vocal response or by analyzing the captured images to detect specific physical motion (e.g., hand motion). For example, when a request for a response is made, the detected person may respond by a vocal yes or a vocal no as to whether assistance is needed. In another embodiment, the user may nod his or her head when assistance is needed and shake his or her head when assistance is not needed. In one embodiment, the user may remain silent or not move when assistance is needed and may wave his or her hand or make other gestures when assistance is not needed.

The monitoring system may be calibrated to the voice of the person to be monitored. The voice recognition patterns may be calibrated to recognize specific commands that are stored in the memory, whether it is for a distress for help or for denying medical assistance. The response from the user may include voice commands instructing the system to perform a particular action that are not pre-programed. For example, the voice command instructions may include initiating a call with a particular person or sending an email or text to a specified person.

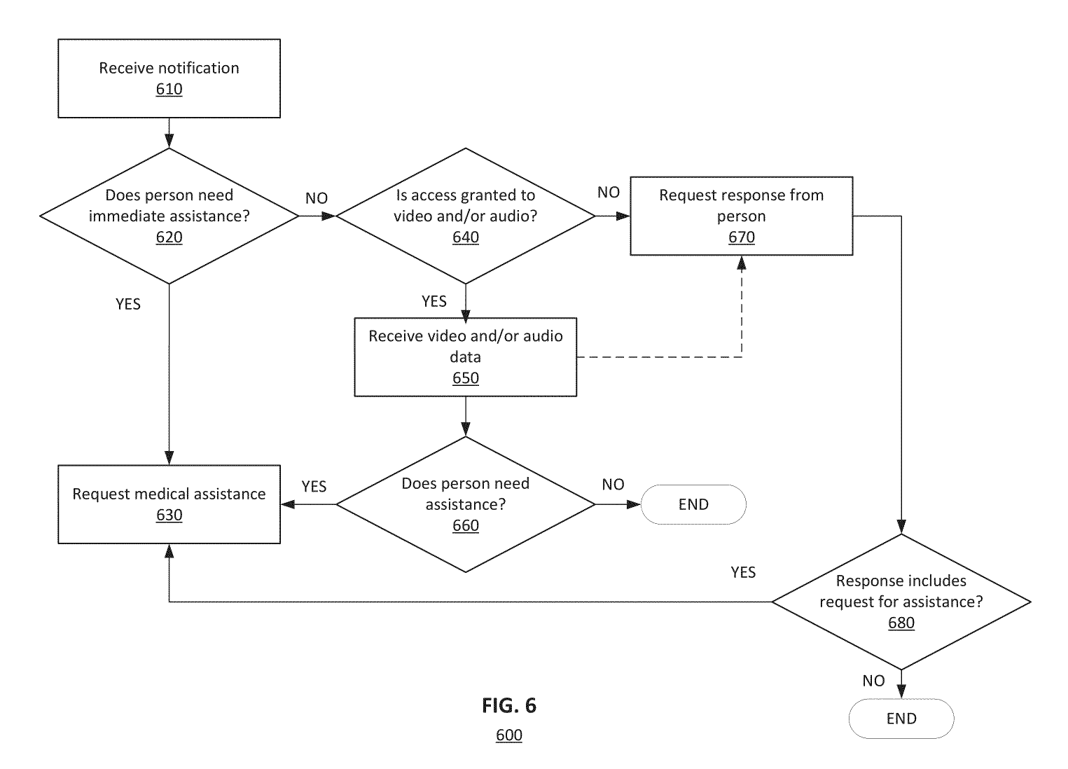

FIG. 6 illustrates a method 600 for responding to a notification of an incapacitated person received from a monitoring system according to an embodiment of the present disclosure. The method may be performed by a processing system at a monitoring center, a designated location (e.g., hospital), and/or a mobile device.

The method 600 may include, receiving a notification from a monitoring system 610, and based on the information provided in the notification, determining whether the person needs immediate assistance 620. The determination may be made based on the information included in the notification. For example, if the notification indicates that a person fell and is not responding, the system may automatically transmit a request for medical assistance 630. The request for medical assistance may include information included in the notification (e.g., identity and location of the incapacitated person).

If access is granted to the video and/or audio data from the monitoring system 640, the video and/or audio data may be processed to determine the severity of the situation. For example, the operator (e.g., emergency operator) may be able to determine the severity of the situation based on the received video and/or audio data. The video and/or audio data may be received 650 and analyzed to determine if the person is in need of medical assistance 660. If it is determined that medical assistance is needed (YES in step 660), the system may transmit the request for medical assistance 630.

If access is not provided to the video and/or audio data (NO in step 640) or if additional information is needed after analyzing the received video and/or audio data, a request for a response from the incapacitated person may be made 670. The request may include a pre-recorded voice request or a request recorded by the operator. In one embodiment, the operator (e.g., emergency personnel) may try to speak to the incapacitated person through a speaker provided as part of the monitoring device or in the vicinity of the monitoring device. Speaking to the incapacitated person may allow the operator to coax the person until they have auditory or vocal capability.

Based on the received response, a determination may be made as to whether a request for medical assistance should be made 680. If it is determined that medical assistance is needed (YES in step 680), the system may transmit the request for medical assistance 630. The operator may initiate the transmission of the request for medical assistance 630 or may manually make the request by calling the appropriate emergency responders to arrive at the residence of the incapacitated person.

While the discussion is generally directed to detecting a single incapacitated person, the embodiments of the present disclosure may be applied to detect multiple incapacitated persons. Information for each of the persons to be monitored may be stored in memory and retrieved when a particular person is determined to be incapacitated. The information stored in memory may include name, medical history, picture (e.g., for face recognition), emergency contact, and/or relative's information. In addition, the embodiments of the present disclosure may be applied to detecting an incapacitated pet.

The communication link (e.g., communication link 130 shown in FIG. 1) may be a network. The network may include: an internet, such as the Internet; an intranet; a local area network (LAN); a wide area network (WAN); an internal network, an external network; a metropolitan area network (MAN); a body area network (BAN); a vehicle area network (VAN); a home area network (HAN); a personal area network (PAN); a controller area network (CAN); and a combination of networks, such as an internet and an intranet. The network may be a wireless network (e.g., radio frequency waveforms, free-space optical waveforms, acoustic waveforms, etc.) and may include portions that are hard-wired connections (e.g., coaxial cable, twisted pair, optical fiber, waveguides, etc.).

Various storage devices (such as the memory shown in FIG. 2) may be utilized herein to store data (including instructions). For example, storage device(s) may include volatile and/or nonvolatile memory (or storage). Nonvolatile memory may include one or more of the following: read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, or other types of nonvolatile machine-readable media that are capable of storing electronic data (e.g., including instructions). Volatile storage (or memory) devices may include random access memory (RAM), dynamic RAM (DRAM), synchronous DRAM (SDRAM), static RAM (SRAM), or other types of storage devices. Also, various components discussed with reference to FIGS. 1 and 2 may communicate with other components through a computer network (e.g., via a modem, network interface device, or other communication devices).

Reference in the specification to “one embodiment” or “an embodiment” means that a particular feature, structure, or characteristic described in connection with the embodiment may be included in at least an implementation. The appearances of the phrase “in one embodiment” in various places in the specification may or may not be all referring to the same embodiment.

Also, in the description and claims, the terms “coupled” and “connected,” along with their derivatives, may be used. In some embodiments of the invention, “connected” may be used to indicate that two or more elements are in direct physical or electrical contact with each other. “Coupled” may mean that two or more elements are in direct physical or electrical contact. However, “coupled” may also mean that two or more elements may not be in direct contact with each other, but may still cooperate or interact with each other.

Thus, although embodiments of the invention have been described in language specific to structural features and/or methodological acts, it is to be understood that claimed subject matter may not be limited to the specific features or acts described. Rather, the specific features and acts are disclosed as sample forms of implementing the claimed subject matter.

Some embodiments of the invention may include the above-described methods being written as one or more software components. These components, and the functionality associated with each, may be used by client, server, distributed, or peer computer systems. These components may be written in a computer language corresponding to one or more programming languages such as, functional, declarative, procedural, object-oriented, lower level languages and the like. They may be linked to other components via various application programming interfaces and then compiled into one complete application for a server or a client. Alternatively, the components maybe implemented in server and client applications. Further, these components may be linked together via various distributed programming protocols.

The above-illustrated software components may be tangibly stored on a computer readable storage medium as instructions. The term “computer readable storage medium” should be taken to include a single medium or multiple media that stores one or more sets of instructions. The term “computer readable storage medium” should be taken to include any physical article that is capable of undergoing a set of physical changes to physically store, encode, or otherwise carry a set of instructions for execution by a computer system which causes the computer system to perform any of the methods or process steps described, represented, or illustrated herein. Examples of computer readable storage media include, but are not limited to: magnetic media, such as hard disks, floppy disks, and magnetic tape; optical media such as CD-ROMs, DVDs and holographic devices; magneto-optical media; and hardware devices that are specially configured to store and execute, such as application-specific integrated circuits (“ASICs”), programmable logic devices (“PLDs”) and ROM and RAM devices. Examples of computer readable instructions include machine code, such as produced by a compiler, and files containing higher-level code that are executed by a computer using an interpreter. For example, an embodiment of the invention may be implemented using Java, C++, or other object-oriented programming language and development tools. Another embodiment of the invention may be implemented in hard-wired circuitry in place of, or in combination with machine readable software instructions.

In the above description, numerous specific details are set forth to provide a thorough understanding of embodiments of the invention. The invention is capable of other embodiments and of being practices and carried out in various ways. One skilled in the relevant art will recognize, however that the invention can be practiced without one or more of the specific details or with other methods, components, techniques, etc. In other instances, well-known operations or structures are not shown or described in details to avoid obscuring aspects of the invention. Also, it is to be understood that the phraseology and terminology employed herein are for the purpose of the description and should not be regarded as limited.

Although the processes illustrated and described herein include series of steps, it will be appreciated that the different embodiments of the present invention are not limited by the illustrated ordering of steps, as some steps may occur in different orders, some concurrently with other steps apart from that shown and described herein. In addition, not all illustrated steps may be required to implement a methodology in accordance with the present invention. Moreover, it will be appreciated that the processes may be implemented in association with the apparatus and systems illustrated and described herein as well as in association with other systems not illustrated.

We claim:

1. A monitoring system comprising:
a camera configured to capture video data, the video data representing a plurality of sequentially captured images of a scene;
a microphone configured to capture audio signals in a vicinity of the camera;
a speaker configured to play audio signals;
memory storing data indicative of conditions identifying an incapacitated person; and
a processing system, comprising at least one processor, coupled to the camera, the microphone, the speaker, and the memory, the processing system being at least configured to:
receive the video data from the camera;
receive the captured audio signals from the microphone;
analyze the images in the video data and/or the captured sounds signals to detect a person and determine whether the detected person satisfies one or more conditions identifying the incapacitated person;
when one or more of the conditions identifying the incapacitated person are satisfied, transmit, to the speaker, an audio signal requesting a response from the detected person;
after the request for the response is transmitted, receive subsequent captured video data from the camera and/or the audio signal captured by the microphone;
analyze the received video data and/or the received audio signal captured by the microphone to determine if the incapacitated person needs medical assistance; and
when it is determined that the incapacitated person needs medical assistance, transmit a notification of the incapacitated person to one or more designated notification locations.

2. The monitoring system of claim 1, wherein when the received video data from the camera and/or the captured audio signals from the microphone indicates that a response to the request is not made by the incapacitated person in a predetermined period of time after transmitting the request for the response, transmitting the notification of the incapacitated person to the one or more designated notification locations.

3. The monitoring system of claim 1, wherein the request for the response is a pre-recorded audio signal that is transmitted to the speaker.

4. The monitoring system of claim 3, wherein after the pre-recorded audio signal is transmitted to the speaker, the processing system is configured to receive the captured audio signal from the microphone and determine whether the received audio signal includes a vocal response of the incapacitated person.

5. The monitoring system of claim 1, wherein the request for the response is a pre-recorded audio signal requesting for the incapacitated person to perform a predefined body motion.

6. The monitoring system of claim 5, wherein after the pre-recorded audio signal is transmitted to the speaker, the processing system is configured to receive the captured video data from the camera and determine whether the images in the received video data include the predefined body motion.

7. The monitoring system of claim 1, wherein one or more conditions identifying the incapacitated person include a body falling and not moving for a predetermined period of time.

8. The monitoring system of claim 1, wherein one or more conditions identifying the incapacitated person include an abnormal heartrate.

9. An incapacitated person monitoring apparatus comprising a processing system, including at least one processor, the processing system being at least configured to:
receive video data from at least one camera, the video data including a plurality of sequentially captured images;
detect a body of a person in the images;
determine whether the detected body of the person in the images satisfies one or more first conditions, stored in memory associated with the incapacitated person monitoring apparatus, identifying an incapacitated person; and
when the body of the person satisfied one or more first conditions, transmitting a notification to one or more notification locations.

10. The incapacitated person monitoring apparatus of claim 9, wherein the one or more first conditions include that motion of the body is stopped for a predetermined period of time.

11. The incapacitated person monitoring apparatus of claim 9, wherein the video data from the camera includes video data captured by a thermographic camera.

12. The incapacitated person monitoring apparatus of claim 11, wherein the one or more first conditions include an abnormal heart rate of the detected body for a predetermined period of time.

13. The incapacitated person monitoring apparatus of claim 11, wherein the one or more first conditions includes that change in temperature of the body, approximated by changes in color of the body in different images captured by the thermographic camera, exceeds a predetermined value.

14. The incapacitated person monitoring apparatus of claim 9, wherein the one or more first conditions include that a magnitude of a motion vector representing motion of the body exceeds a predetermined threshold.

15. The incapacitated person monitoring apparatus of claim 9, wherein the processing system is further configured to, when the body of the person in the images does not satisfy one or more first conditions, determine whether the body of the person detected in the images satisfies one or more second conditions, stored in the memory, identifying a person that is potentially incapacitated; and when the body of the person satisfied one or more second conditions, transmit a request for a response from the person in the images.

16. The incapacitated person monitoring apparatus of claim 15, wherein the processing system is further configured to analyze the images, received after transmitting the request, to determine whether the incapacitated person responded within a pre-determined period of time; and when the response is not detected in the images within the pre-determined period of time, transmit a notification to one or more notification locations stored in the memory.

17. The incapacitated person monitoring apparatus of claim 15, wherein the request for a response includes a description of a body motion and the processing system is further configured to analyze the images, received after transmitting the request, to determine whether the incapacitated person responded with the body motion included in the request for the response; and when the response with the body motion included in the request for the response is not detected in the images, transmit a notification to one or more notification location stored in the memory.

18. A computer implemented method for detecting an incapacitated person, the method comprising:
receiving, from one or more cameras, video data including a plurality of sequentially captured images;
detecting a person in the images;
determining a motion vector representing motion of the detected person;
determining a heart rate of the person; and
when it is determined that magnitude of the motion vector is greater than a predetermined value or it is determined that the heart rate of the body is abnormal, automatically transmitting a notification to one or more notification locations requesting medical assistance.

19. The computer implemented method of claim 18, wherein the video data is received from a thermographic camera and the heart rate of the person is determined by analyzing the plurality of images in the video data received from the thermographic camera.

20. The computer implemented method of claim 19, further comprising:
determining changes in body temperature of the detected person by comparing a plurality of sequentially captured images received from the thermography camera; and
when the changes in the body temperature exceeds a preset range, automatically transmitting a notification to the one or more notification locations requesting for medical assistance.
